(19) United States
(12) Patent Application Publication
(10) Pub. No.: US 2006/0263344 A1
(43) Pub. Date: Nov. 23, 2006

(54) Novel probiotic compositions and methods of using the same

(76) Inventors: Mitchell Skop, Closter, NJ (US); Dilip Chokshi, Parsippany, NJ (US)

Correspondence Address: Ronald J Baron;Hoffman & Baron, 6900 Jericho Turnpike, Syosset, NY, 11791, US

(21) Appl. No.: 10/551,665

(22) PCT Filed: Apr. 1, 2004
(86) PCT No.: PCT/US04/10164

§ 371 Date: Jun. 15, 2006

Related U.S. Application Data
(60) Provisional application No. 60/459,789, filed on Apr. 2, 2003.

(51) Int. Cl. A61K 9/68; A61K 36/28; A61K 35/74

(52) U.S. Cl. 424/93.45; 424/440; 424/764

(57) ABSTRACT

The invention provides a soft, chewable confection obtained without using high heat or dehydration. In one embodiment, the confection contains an active ingredient which consists essentially of an effective amount of at least one probiotic organism. In another embodiment, the confection consists essentially of an effective amount of at least one probiotic organism and an effective amount of at least one prebiotic. In a further embodiment, the confection consists essentially of an effective amount of at least one probiotic organism and a nutrient contained in a glycoprotein matrix, or an enzyme, or both. In yet another embodiment, the confection consists essentially of at least one probiotic organism, at least one prebiotic, and a nutrient contained in a glycoprotein matrix, or an enzyme, or both.

### BACKGROUND OF THE INVENTION

The present invention relates to novel, ingestible formulations containing probiotic organisms, optionally with prebiotics. More specifically, the present invention relates to probiotic organisms, optionally with prebiotics, administered in a soft, chewable confection.

“Probiotic” is a compound Latin and Greek word that means “favorable to life.” Probiotic organisms are live microbes that are beneficial to the health of individuals. One of the major health benefits of ingesting probiotic organisms is to supplement the natural flora of the gastrointestinal tract with additional bacteria.

Bacteria that occupy the gastrointestinal tract have been shown to play a number of vital roles in maintaining function and overall physiological health. The growth and metabolism of the diverse bacterial species that occupy the gastrointestinal tract depend on materials available to them. Most of these needed materials are derived from the diet.

In addition to supporting the gastrointestinal tract, it has been shown that supplementation with probiotic organisms may be effective in treating a vast array of conditions, including cancer, dermatitis, allergies, and upper respiratory infections. See, e.g. Rafter, J J., “Scientific Basis of Biomarkers and Benefits of Functional Foods for Reduction of Disease Risk: Cancer,” Br. J. Nutr. November 2002;88 Suppl 2:S219-24; Rosenfeldt, V. et al., “Effect of Probiotic Lactobacillus Strains in Children with Atopic Dermititis,” J. Allergy Clin. Immunol February 2003;111(2):389-95; Kalliomaki, M. et al., “Role of Intestinal Flora in the Development of Allergy,” Curr. Opin. Allergy Clin. Immunology February 2003;3(1):15-20; and Mercenier, A., et al. “Probiotics as Biotherapeutic Agents: Present Knowledge and Future Prospects,” Curr. Pharm. Des. 2003;9(2):175-91.

Examples of probiotic organisms include lactic acid producing bacteria such as Lactobacillus, Lactococcus and Bifidobacterium, and other bacteria such as Streptococcus.

The probiotic organisms which are found in foods and nutritional supplements are not normally found in the gastrointestinal tract. In fact, the intestinal environment is often a hostile environment for these foreign bacteria. Therefore, the bacteria consumed in probiotic products do not colonize in the intestine. Rather, they are flushed through and eliminated quickly from the body. Since probiotic organisms do not permanently colonize the host, they need to be ingested regularly in order to gain desired health benefits.

“Prebiotics” are foods or nutrients that are used by specific bacteria that can be added to the diet to increase the chances of these particular bacteria growing and thriving in the intestine. Examples of prebiotics include inulin, chicory and honey. Inulin is a polysaccharide found in the roots of various composite plants and yields fructose when hydrolyzed.

The best known example of a food containing probiotic organisms, and sometimes prebiotics, is yogurt. Aside from yogurt and other dairy preparations (e.g. kefir), powder, tablet and liquid formulations that contain probiotic organisms are known. See, e.g. U.S. Patent Application 20030032659 to Farmer; U.S. Pat. No. 6,511,685 to Weissmahr.

However, problems exist with the currently available probiotic formulations. Of importance is the fact that probiotic organisms are extremely heat sensitive. In order to prepare powder or tablet formulations, dehydration using high heat is necessary. Exposure to heat, especially high heat, will destroy the probiotic organisms. Therefore, it is doubtful that powder or tablet formulations contain effective amounts of live probiotic organisms, if any.

Lyophilized, i.e. freeze dried, probiotic organisms are also available. Freeze dried bacteria are in anabiotic state. The need to wet the microorganism before administration, in order to reinstate vitality, is a disadvantage since many bacteria will not survive rehydration. Moreover, the surviving organisms, if any, are not immediately metabolically active, and cannot survive the extreme, acidic conditions of the stomach.

Liquid formulations containing probiotic organisms must be kept refrigerated to keep the organisms viable. Liquid probiotic formulations have a very short shelf life. Unfortunately, many of the probiotics in liquid formulations are killed during transportation to the retailer, or while the product stands on the shelf in a store. Moreover, there are compliance problems with liquid formulations due to unacceptable taste quality.

As mentioned above, probiotic organisms get flushed through the intestine and must be consumed regularly to gain desired health benefits. The needed compliance is rarely met because the currently available products have an unacceptable taste, and are inconvenient.

Dairy products containing probiotic organisms, such as yogurts and kefirs, also have problems. For example, many individuals taking probiotic organisms are suffering from gastrointestinal problems (e.g. antibiotic use, gastritis, irritable bowel syndrome, gas), and are trying to avoid consuming dairy products. Dairy products are known to contribute solely or collectively to gastrointestinal problems.

In addition, most yogurt and kefir products are heavily sweetened (e.g. sugars, fruit syrups) to mask the sour taste of the cultured dairy product. The addition of such sweeteners is undesirable to weight conscious consumers and diabetics. Importantly, many individuals taking probiotics are suffering from a yeast infection caused by conditions such as antibiotic use or diabetes, and are trying to avoid sugars. Sugars aggravate both yeast infections and diabetes.

Hence, there is a long felt need for a formulation containing live probiotic organisms, and optionally prebiotics, that can be easily transported, taken regularly, has a reasonable shelf life, is easily digested, and does not contain excessive sweeteners.

It is thus an object of the present invention to provide a probiotic formulation, optionally with prebiotics, that overcomes the above deficiencies.

### SUMMARY OF THE INVENTION

As a result of the present invention, Applicants have surprisingly discovered that probiotic organisms have increased viability, extended shelf life and improved compliance among users, when delivered in a soft, chewable confection according to the invention.

The invention provides a soft, chewable confection containing an active ingredient, wherein the active ingredient consists essentially of an effective amount of at least one probiotic organism, wherein the confection is obtained without using high heat or dehydration.

Another embodiment of the invention provides a soft, chewable confection containing an active ingredient, wherein the active ingredient consists essentially of an effective amount of at least one probiotic organism and at least one prebiotic, wherein the confection is obtained without using high heat or dehydration.

The invention also provides a soft, chewable confection containing an active ingredient, wherein the active ingredient consists essentially of an effective amount of at least one probiotic organism and a nutrient contained in a glycoprotein matrix, or an enzyme or both, wherein the confection is obtained without using high heat or dehydration.

In yet another embodiment, a soft, chewable confection containing an active ingredient, wherein the active ingredient consists essentially of an effective amount of at least one probiotic organism, at least one prebiotic, and a nutrient contained in a glycoprotein matrix, or an enzyme, or both, wherein the confection is obtained without using high heat or dehydration, is provided.

In one embodiment, the soft, chewable confection contains no dairy.

Preferably, the effective amount of probiotic organism is from approximately 1×10⁵ to approximately 2×10⁵ organisms per confection. More preferably, the effective amount of probiotic organism is at least 2×10⁵ organisms per confection.

In yet another embodiment, the probiotic organism is selected from the group consisting of Lactobacillus, Bifidobacteria, Lactococcus, and Streptococcus, and combinations thereof. Preferably, the probiotic organism is Lactobacillus acidophilus, Streptococcus thermophilus, or Lactobacillus bifidus, or combinations thereof.

Another embodiment of the invention provides a soft, chewable confection wherein the prebiotic is inulin, chicory or honey. Preferably, the prebiotic is honey.

In one embodiment, the nutrient contained in a glycoprotein matrix is coenzyme Q10, 1-carnitine, or alpha lipoic acid, or a combination thereof. Preferably, the nutrient contained in a glycoprotein matrix is coenzyme Q10.

In an alternate embodiment, the enzyme is papain or lactase, or a combination thereof. Preferably, the nutrient contained in a glycoprotein matrix is coenzyme Q10, and the enzyme is a combination of papain and lactase.

In yet another embodiment, a method for enhancing immune function of a host in need thereof is provided. The method comprises administering an effective amount of a soft, chewable confection containing an active ingredient, wherein the active ingredient consists essentially of an effective amount of at least one probiotic organism and optionally a prebiotic and a nutrient contained in a glycoprotein matrix, or an enzyme, or both, wherein the confection is obtained without using high heat or dehydration.

In an alternate embodiment, a method for providing probiotic organisms to a lactose intolerant host in need thereof is provided. The method comprises administering an effective amount of a soft, chewable confection containing an active ingredient, wherein the active ingredient consists essentially of an effective amount of at least one probiotic organism, wherein the confection is obtained without using high heat or dehydration, and wherein the confection contains no dairy.

The invention also provides a method for providing probiotic organisms to a lactose intolerant host in need thereof. The method comprises administering an effective amount of a soft, chewable confection containing an active ingredient, wherein the active ingredient consists essentially of an effective amount of at least one probiotic organism, and an effective amount of lactase, wherein the confection is obtained without using high heat or dehydration.

### DETAILED DESCRIPTION OF THE INVENTION

The present invention relates to novel formulations containing probiotic organisms, and optionally prebiotics. Specifically, the present invention is for a soft, chewable confection containing an active ingredient, wherein the active ingredient consists essentially of an effective amount of a probiotic organism.

Probiotic Organisms

“Probiotic organisms” are live microorganisms that beneficially affect the health of a host. The benefits to the health of the host include, but are not limited to, improving the microbial balance of the intestines. Other beneficial effects to the host include, for example, enhancing the immune system, stimulation of phagocytotic activity, stimulation of interferon, reduction of hypertension, decrease in the risk of colon cancer, increase in antimicrobial activity and immunomodulating effects, reduction of hypercholesterolemia, and treatment of cancer and dermatitis.

A probiotic organism of the present invention includes any microorganism that exhibits a beneficial effect on a host, and is non-pathogenic. By way of example, and not of limitation, many examples of suitable bacteria have been identified and are described herein as probiotic organisms. Although, it should be noted that the present invention is not to be limited to currently-classified bacterial species insofar as the purposes and objectives as disclosed.

A probiotic organism of the present invention includes any lactic acid producing bacteria which includes non-pathogenic members of the Bacillus genus, Bifidobacterium genus, Lactococcus genus, Streptococcus genus, and Lactobacillus genus.

Exemplary Bacillus species include, but are not limited to, Bacillus coagulans and Bacillus brevis. Exemplary Bifidobacterium species include, but are not limited to, Bifidobacterium adolescentis, Bifidobacterium animalis, Bifidobacterium bifidum, Bifidobacterium bifidus, Bifidobacterium brevis, Bifidobacterium infantis, Bifidobacterium longum, and any genetic variant thereof.

Exemplary Lactococcus species includes, but are not limited to, Lactococcus lactis. Exemplary Streptococcus species includes, but are not limited to, Streptococcus thermophilus.

Exemplary Lactobacillus species include, but are not limited to, Lactobacillus acidophilus, Lactobacillus casei, Lactobacillus rhamnosus, Lactobacillus, plantarum, Lactobacillus reuteri, Lactobacillus bulgaricus, and Lactobacillus delbruekii, Lactococcus brevis, and Lactobacillus heveticus.

Preferably, the probiotic in the confection is selected from the group consisting of Lactobacillus, Bifidobacterium, Streptococcus, and Lactococcus and combinations thereof. Even more preferred, the probiotic is Lactobacillus acidophilus, Streptococcus thermophilus, or Lactobacillus bifidus, or a combination thereof.

The combination of probiotics used will depend on the health of the host and the benefit desired. A skilled artisan can easily determine which probiotic(s) should be utilized. For example, Bifidobacteria are known to be involved in resisting colonization of pathogens in the large bowel. Use of a combination of Bifidobacterium bifidum and Streptococcus thermophilus has been shown to reduce rotavirus shedding and episodes of diarrhea in children.

Lactobacilli is useful in treating diarrhea infections such as, for example, pseudomembranous colitis. Lactobacilli, Bifidobacteria, and Streptococci are used prophalactically to prevent traveler's diarrhea caused by enterdtoxigenic Escherichia coli.

Probiotic organisms interact with the immune system at many levels, including cytokine production, mononuclear cell proliferation, macrophage phagocytosis and killing, modulation or autoimmunity, and immunity to bacterial and protozoan pathogens.

For example, Bifidobacterium breve enhances production of antibodies against food allergens and pathogens. Lactobacillus acidophilus and Bifidobacterium bifidum reduce colonic inflammatory infiltration.

An effective amount of probiotic organisms is any amount that provides a benefit to the health of the host. Preferably, the effective amount of probiotics is from approximately 1×10⁵ to approximately 2×10⁵ organisms per confection. More preferably, the effective amount of probiotics is at least 2×10⁵ organisms per confection.

Prebiotics

In one embodiment of the invention, the active ingredient is an effective amount of at least one probiotic organism and at least one prebiotic. “Prebiotic” is defined as a non-digestible food ingredient that selectively supports (e.g. stimulates the growth or activity, or both), of beneficial bacteria, e.g. probiotic organisms.

A prebiotic of the present invention includes any oligosaccharide that provides support, as described above, to beneficial bacteria. For example, suitable prebiotics include low molecular weight carbohydrates such as inulins and fructooligosaccharides. Inulins and fructooligosaccharides occur naturally in artichokes, onions, chicory, garlic, leeks, and to a lesser extent, in cereals.

Honey is also a suitable prebiotic, as well as other oligosaccharides including raffinose and stachyose which are the major carbohydrates in beans and peas.

Preferably, the prebiotic is inulin, chicory or honey, or a combination thereof. More preferably, the prebiotic is honey.

The amount of prebiotic per confection is any amount that effectively provides support to probiotic organisms in the host. Preferably, the amount of prebiotic is approximately 50 to 500 mg per confection. More preferably, the amount of prebiotic is approximately 100 to 250 mg per confection.

Nutrient Contained in a Glycoprotein Matrix

In another embodiment of the invention, the active ingredient of the confection is an effective amount of at least one probiotic organism, at least one prebiotic and a nutrient contained in a glycoprotein matrix, or an enzyme, or both.

A “nutrient contained in a glycoprotein matrix,” according to the invention, is any substance that can be metabolized by an organism to give energy and build tissue, that is enrobed or bound by a glycoprotein matrix.

A “glycoprotein matrix” is a network of glycoproteins that is available to be bound to a nutrient by allowing one or more microorganisms to ferment, in the presence of the nutrient. As a result of the fermentation, glycoproteins are secreted from the microorganisms. These glycoproteins are mainly extracellular and, therefore, are available to be bound to a nutrient. A suitable glycoprotein matrix, and processes for binding a nutrient to a glycoprotein matrix, are disclosed in co-pending U.S. patent application Ser. No. 09/757,222 filed Jan. 9, 2001 which is a continuation-in-part of U.S. patent application Ser. No. 09/906,576 filed Jul. 16, 2001, both of which are incorporated by reference, in their entirety.

According to the invention, the nutrient preferably is nutrient that becomes depleted in a host that has undergone, is undergoing or plans to undergo treatment with an antibiotic.

In addition, suitable nutrients include nutrients that are decreased or needed at increased levels, in a host that suffers from an impaired immune system, e.g. diabetes mellitus, cancer, etc. Suitable nutrients are also nutrients that are unstable, e.g. heat sensitive, such as, for example, co-enzyme Q10, amino acids, enzymes and proteins.

Preferred nutrients include co-enzyme Q10, 1-carnitine and alpha lipoic acid, or combinations thereof. Preferably, the nutrient is co-enzyme Q10.

Coenzyme Q10

“Coenzyme Q₁₀” (hereinafter “CoQ₁₀”), is a benzoquinone compound synthesized naturally in the body. The “Q” and the “10” in the name refer to the quinine chemical group and the ten isoprenyl chemical subunits, respectively. CoQ₁₀ is essentially a vitamin-like substance found in small amounts in a wide variety of foods, and is synthesized in all tissues. The biosynthesis of CoQ₁₀ from the amino acid tyrosine is a multi-stage process requiring eight vitamins and several trace elements. Co-enzymes are co-factors upon which comparatively large and complex enzymes depend for their function. CoQ₁₀ is the co-enzyme for at least three mitochondrial enzymes (complexes I, II and III) as well as enzymes in other parts of the cell.

Mitochondrial enzymes of the oxidative phosphorylation pathway are essential for the production of adenosine triphosphate (ATP), upon which all cellular functions depend. CoQ₁₀ plays a critical role in the sequential transfer of electrons in the mitochondrion.

In addition to electron transport in the mitochondrion, CoQ₁₀ has also been found to be important in the prevention of cellular-free radical damage, oxygenation at the cellular level, as well as other benefits. CoQ₁₀ is known to stimulate immune function.

Studies have shown that a decrease in CoQ₁₀ levels by 25% results in an inability of the body to produce enough cellular energy to remain healthy. A decline of 75% in CoQ₁₀ can be fatal.

It is well known that lipid lowering agents such as the “statins” (e.g. lovastatin, pravastatin and simvastatin) and gemfibrozil, as well as oral agents that lower blood sugar such as glyburide and tolzamide, cause a decrease in serum levels of CoQ₁₀.

It is known to administer CoQ₁₀ for the treatment or prevention of various ailments. Thus, the confection contains an amount of the glycoprotein matrix-containing CoQ₁₀ such that a sufficient amount of CoQ₁₀ is administered to achieve the desired result. Such amounts can be determined by one skilled in the art.

L-Carnitine

“L-carnitine” is an amino acid that is synthesized in the human body, mainly in the liver and kidneys, from essential amino acids, lysine and methionine. By “L-carnitine” it is meant to include any ester of L-carnitine, such as, for example, acetyl-L-carnitine. L-carnitine's main function in the body is to transport long-chain fatty acids into the mitochondria.

Clinical studies have shown that L-carnitine is effective in treating conditions such as Alzheimer's Dementia, depression, HIV infection, diabetes neuropathy, cataracts, cerebral ischemia and reperfusion.

It is known to administer L-carnitine for the treatment or prevention of various conditions as discussed above. Thus, the confection contains an amount of the glycoprotein matrix-containing L-carnitine such that a sufficient amount of L-carnitine is administered to achieve the desired result. Such amounts can be determined by one skilled in the art.

Alpha Lipoic Acid

“Alpha lipoic acid” (also known as lipoic acid or thioctic acid) is a sulf-containing vitamin-like antioxidant. Alpha lipoic acid is produced naturally in the body and found in food sources such as liver, brewer's yeast and potatoes.

Alpha lipoic acid has a dual role in human health; it is a powerful antioxidant and a key component for producing cellular energy. As an antioxidant, alpha lipoic acid extends and enhances the effect of other antioxidants. In its metabolic role, alpha lipoic acid is a fundamental coenzyme in vital reactions that lead to the production of cellular energy (ATP).

Generally, sufficient levels of alpha lipoic acid are produced in the body or acquired from food. However, certain diseases, environmental conditions, and age can cause a deficiency in lipoic acid, and thus the body often does not make enough to meet all its metabolic and antioxidant needs.

Supplementation with alpha lipoic acid has been shown to improve energy metabolism and prevent recognizable disease. This is particularly applicable in people with conditions such as, for example, diabetes and HIV.

Alpha lipoic acid has been indicated for normalizing blood sugar levels. It is believed that alpha lipoic acid helps control blood sugar by facilitating the conversion of sugar to energy. Alpha lipoic acid has also been shown to reduce glycation (i.e. glycosylation), which is the process in which proteins react with excess glucose resulting in free radical damage.

Alpha lipoic acid has also been indicated in the treatment of HIV infection. During HIV infection, lymphocytes lose their ability to make and transport glutathione. Glutathione is a major cellular antioxidant that acts to prevent HIV viral replication. Alpha lipoic acid is a facilitator of glutathione production. Clinical studies have shown that supplementation with alpha lipoic acid increased total glutathione in HIV infected individuals.

Alpha lipoic acid has also been shown to protect against cancer and to provide beneficial protection to cancer patients. For example, lipoic acid protects a complex called Nuclear Factor kappa-B and prevents it from activating oncogenes.

It is known to administer alpha lipoic acid for the treatment or prevention of various ailments as discussed above. Thus, the confection contains an amount of the glycoprotein matrix alpha lipoic acid such that a sufficient amount of alpha lipoic acid is administered to achieve the desired result. Such amounts can be determined by one skilled in the art.

Increased Bioavailability of Nutrient

Binding a nutrient to a glycoprotein matrix increases the bioavailability, bioactivity and stability of the nutrient. For example, it has been discovered that binding CoQ₁₀ to a glycoprotein matrix increases the bioactivity of the CoQ₁₀. As mentioned above, it is known that CoQ₁₀ can have an antioxidative effect. As described below in Example 2, CoQ₁₀ bound to a glycoprotein matrix have antioxidant activity approximately 20 times that of commercial CoQ₁₀.

In addition, it has been discovered that binding the nutrient to a glycoprotein matrix can increase the stability of the nutrient. For example, CoQ₁₀ can deteriorate when exposed to air. By binding the CoQ₁₀ with a glycoprotein matrix, this deterioration is decreased.

As demonstrated below in Example 1, the CoQ₁₀ contained in a glycoprotein matrix lost only half as much CoQ₁₀ over 36 days compared to commercial CoQ₁₀ when exposed to open air at 50° C.

CoQ₁₀ L-carnitine, and alpha lipoic acid are all heat sensitive nutrients. By “heat sensitive,” it is meant that the nutrient is degraded partially or totally upon exposure to heat, such as the heat that is utilized during conventional methods of producing nutritional supplements.

It is believed that a confection containing probiotics, optionally with prebiotics, and CoQ₁₀, L-carnitine, or alpha lipoic acid, or a combination thereof, enrobed or bound by a glycoprotein matrix, and delivered in a confection as described below, will provide a synergistic effect that will benefit a host.

Not being bound by theory, it is believed that the combination of probiotics, optionally with prebiotics, and CoQ₁₀, L-carnitine, or alpha lipoic acid, or a combination thereof, enrobed or bound by a glycoprotein matrix, and delivered in a confection as mentioned above and further described below, will provide superior health benefits to the host.

The addition of an enzyme to the confection, in combination with a glycoprotein matrix, or by itself, aids in the digestion of the confection.

Enzymes

In a preferred embodiment, an enzyme is added to the confection. Suitable enzymes include, but are not limited to, proteolytic enzymes such as papain, bromelain, pepsin or fungal protease. Other enzymes such as lactase, the enzyme necessary to break down lactose, are also suitable enzymes.

Without being bound by theory, it is believed that the proteolytic enzymes improve the digestibility of the confection in the host. The amount of proteolytic enzyme utilized is any amount sufficient to provide assistance in the digestion of the confection, without affecting the integrity of the confection, e.g. diminishing the viability of the probiotics. This amount will vary depending upon the confection. Typically, approximately 1 to 50 g of proteolytic enzyme will be added per confection.

The amount of lactase utilized is any amount sufficient to aid in the digestion of lactose, without affecting the integrity of the confection, e.g. diminishing the viability of the probiotics. Such an amount can be determined by one of ordinary skill in the art.

Other suitable enzymes include, for example, amylase, protease, lipase and cellulose.

Additives

If desired, appropriate additives may be included in the confection. The amount of additive is the amount necessary to obtain the desired beneficial result, without diminishing the viability of the probiotic organisms. The amounts of such additives can be determined by one skilled in the art.

Such additives may include, for example, stabilizers. Stabilizers are substances that improve the stability of the probiotic organism, prebiotic, and/or nutrient. For example, one class of stabilizers is bioflavanoids. Preferred bioflavanoids include hesperidin, quercitin and rutin. Since these bioflavanoids are naturally obtained, commercially available bioflavanoids very often will include additional materials such as fibers or cellulose. The active portion, e.g. hesperidin, quercitin, or rutin, will make up a percentage of the bioflavanoid. The active ingredient in the bioflavanoid will usually vary between approximately 10-60%.

Other additives can be added which, for example, improve the viability of the microorganisms that produce the glycoprotein matrix or increase the yield of glycoprotein that becomes bound to the nutrient. For example, salts can be added in order to increase the viability of the microorganism. Such salts include, but are not limited to, calcium carbonate, ammonium sulfate, and magnesium sulfate. Calcium carbonate is preferred. The amount of salt added should be sufficient to obtain the desired result of improving the viability of the organism, as is known in the art.

In a preferred embodiment, the confection contains no dairy. “Dairy” includes any food product derived from the milk of an animal such as, for example, a cow.

The Confection

The soft, chewable confection of the present invention is any chewable confection that has a nougat candy consistency. For example, the confection imparts a soft, yet unsticky chew texture. Such a confection can be obtained by any known method, so long as the use of high heat (e.g. greater than 60° C.), excessive moisture (e.g. an amount that requires dehydration) and dehydration processes are avoided.

A suitable method for producing such a confection is disclosed in U.S. Pat. No. 6,517,886, assigned to Biovail Corp Int'l, which is herein incorporated by reference in its entirety.

By providing the probiotic organisms, and optionally prebiotics, and a nutrient contained in a glycoprotein matrix, or an enzyme, or both, in a soft, chewable confection that is obtained without the use of high heat, excessive moisture and dehydration, will increase compliance among users.

For example, the confection will provide the desired amounts of the active ingredients in a volume that is substantially less than, for example, a 6 to 8 ounce serving of yogurt. Moreover, the confection can be easily consumed several times per day if needed.

Importantly, the confection of the present invention will provide as much or more probiotic organisms than a 6 to 8 ounce serving of yogurt with substantially less calories than yogurt.

The confection is dispensed as individually wrapped pieces or in a scored bar that can be broken off and consumed as an individual piece. Storage and transportation of the confection is much improved over that of a liquid or yogurt preparation.

Methods of Using the Confection

The confection of the present invention is effectively used to enhance the immune system of a host in need thereof. Hence, in another embodiment of the invention, a method for enhancing the immune system of a host in need thereof is provided. The method comprises administering an effective amount of a confection containing an active ingredient, wherein the active ingredient consists essentially of an effective amount of at least one probiotic organism, optionally with a prebiotic and/or a nutrient contained in a glycoprotein matrix, and/or an enzyme, as described above. The confection is obtained without the use of high heat or dehydration.

For example, by “enhancing the immune system” it is meant that the host has an increased ability to recover from illness. Enhancing the immune system also means that the host experiences a shortened period of illness and/or less severe symptoms of illness.

Optimal doses of the confection can be determined by one skilled in the art based on a number of parameters including, for example, age, sex, weight, condition being treated, the severity of the condition, and the active ingredients utilized. Generally, the effective amount of the confection is approximately one to three confections per day.

A host in need thereof is any host that can benefit from enhancing their immune system as described above. Examples of a host in need thereof include a diabetic or an HIV-infected individual.

In an alternate embodiment, the host in need thereof is lactose intolerant, i.e. cannot properly digest dairy products such as milk. Moreover, the present invention is for a method for providing probiotic supplementation to a lactose intolerant host in need thereof by administering a soft chewable confection that contains an active ingredient, wherein the active ingredient consists essentially of an effective amount of at least one probiotic organism, optionally with a prebiotic, and/or a nutrient contained in a glycoprotein matrix, and/or an enzyme, wherein the confection does not contain dairy, or wherein the confection contains a sufficient amount of lactase. The confection is obtained without the use of high heat or dehydration.

A sufficient amount of lactase for this embodiment of the invention is an amount that is effective to reduce or eliminate the symptoms associated with lactose intolerance. The symptoms of lactose intolerance include, for example, stomach pain, stomach distention, flatulence and diarrhea.

In a preferred embodiment the host is a mammal. Mammals include, for example, humans, as well as pet animals such as dogs and cats, laboratory animals such as rats and mice, and farm animals such as horses and cows. Humans are most preferred.

The following examples illustrate the increased bioactivity and stability of nutrient contained in a glycoprotein matrix. CoQ₁₀ is used as an exemplary nutrient, and it is believed that the same improvements to the bioactivity and stability of CoQ₁₀ will be observed with any other suitable nutrient.

### EXAMPLE 1

The bioactivity of a glycoprotein matrix containing CoQ₁₀ was examined relative to commercially available CoQ₁₀ (USP).

A weighed portion (50-500 mg) of solid sample of CoQ₁₀ contained in a glycoprotein matrix was mixed with 5 ml of 50% methanol/water and heated at 90° C. in a plastic screw-capped tube with intermittent shaking for 2 hours to determine the unconjugated (“free”) phenols present. Another weighed portion of the same sample was heated with 5 ml of 1.2 M HCl in 50% aqueous methanol for 2 hours at 90° C. to measure the unconjugated plus conjugated (“total”) phenols. The extracts, each done in duplicate, were then filtered with a 0.45 μm filter and stored at −20° C. until assay. Values for free polyphenols and total phenols for commercial CoQ₁₀ are known.

The phenol content in the extracts was measured by the Folin-Cocialteu reagent (Sigma Chemical Co., St. Louis, Mo.) using catechin (Sigma) as a standard. A blank, catechin standards and samples were added to the Folin reagent in a cuvette and after 20 minutes the color was measured at 720 nm vs. a blank.

Quality of antioxidant activity was determined in a dose-response assay of the IC₅₀ value, i.e. the concentration of phenols in the extract to inhibit 50% of the oxidation of lower density lipoproteins (LDL+VLDL). This model is an in vitro model of atherosclerosis where the initial step is the oxidation of the lower density lipoproteins, i.e. the “bad” cholesterol. LDL+VLDL is isolated from the plasma of normocholesterolemic humans using an heparin-agarose affinity column (H-6508, Sigma). Extracts of antioxidants were added in duplicate at various concentrations (typically 0.05 to 15 μM) to LDL+VLDL (70 μg/ml of protein as measured vs. albumin standard with Coomasie Blue, Sigma). 25 μM of the oxidant cupric ion was then added, the solution made to a total volume of 400 μL with phosphate buffered saline, pH 7.4 (Sigma) and the solution left at 37° C. for 6 hours.

The amount of lipid peroxides was measured using thiobarbituric acid and fluorometry. The % of inhibition of lipid peroxide formation was calculated vs. a control with no added antioxidants. The IC₅₀ value in μM units was then calculated.

The amount of CoQ₁₀ in the composition of the invention was determined by HPLC using UV detector, C18 column (Perkin Elmer Pecosil 5, 15 cm) and a solvent of 75% methanol and 25% isopropanol.

The results are set forth in Table 1 below. The higher the 1/IC₅₀ value, the better the quality of antioxidants.

The methods used are further described in: Vinson, J. A., and Hontz, B. A. Phenol antioxidant index: comparative antioxidant effectiveness of red and white wines, J. Agric. Food Chem., 1995, 43, 401-403; Vinson, J. A., Jang, J., Dabbagh, Y. A., Serry, M. M., and Cai, S. Plant polyphenols exhibit lipoprotein-bound antioxidant activity using an in vitro model for heart disease. J. Agric. Food Chem., 1995, 43, 2798-2799; and Steinberg, D., Parthasarathy, S., Carew, T. E., Khoo, J. C., and Witzum, J. L. Beyond cholesterol: modification of low density lipoprotein that increases its atherogenicity. New Eng J. Med., 1989, 320, 915-924; all of which are incorporated herein by reference.

TABLE 1

| | IC50 | |
|---|---|---|
| SAMPLE | (uM) | 1/IC50 |
| CoQ₁₀ contained in a | 0.064 | 15.6 |
| glycoprotein matrix contains | (based on CoQ₁₀ conc.) | |
| 8.4% CoQ₁₀ | | |
| CoQ₁₀ (USP) | 1.33 | 0.751 |

The results demonstrate that the CoQ₁₀ contained in a glycoprotein matrix has an antioxidant activity that is 20 times better than commercially available CoQ₁₀.

### EXAMPLE 2

The stability of CoQ₁₀ contained in a glycoprotein matrix was examined.

100 mg of USP CoQ₁₀ (Sigma) and CoQ₁₀ contained in a glycoprotein matrix from Example 1 was placed in a 10 ml beaker in a 50° C. oven open to the air. The amount of CoQ₁₀ remaining was analyzed by HPLC using a C18 column (Perkin Elmer Pecosil 5, 15 cm) and a solvent of 75% methanol and 25% isopropanol. The results are set forth below in Table 2.

TABLE 2

| | | Loss of CoQ₁₀ | Loss of CoQ₁₀ |
|---|---|---|---|
| | | after 36 days at | after 72 days at |
| | Loss of | 50° C. (equivalent | 50° C. (equivalent |
| | CoQ₁₀ | to 3 months at | to 6 months at |
| Sample | at 0 days | room temperature) | room temperature) |
| USP CoQ₁₀ | 0% | 6.8% | 16.8% |
| CoQ₁₀ contained | 0% | 3% | 14.7% |
| in a | | | |
| glycoprotein | | | |
| matrix | | | |

After 36 days, the glycoprotein matrix-containing CoQ₁₀ lost only half as much as the commercial CoQ₁₀ material, i.e. 3% vs. 6.8%. After 72 days, the glycoprotein matrix-containing CoQ₁₀ lost 14.7% of its CoQ₁₀ vs. 16.8% CoQ₁₀ lost with the commercial sample. Therefore, the results show that the CoQ₁₀ contained in a glycoprotein matrix has increased stability.

While there have been described what are presently believed to be the preferred embodiments of the invention, those skilled in the art will realize that changes and modifications may be made thereto without departing from the spirit of the invention, and it is intended to claim all such changes and modifications as fall within the true scope of the invention.

1. A soft, chewable confection containing an active ingredient, wherein the active ingredient consists essentially of an effective amount of at least one probiotic organism, wherein the confection is obtained without using high heat or dehydration.

2. A soft, chewable confection according to claim 1, wherein the confection contains no dairy.

3. A soft, chewable confection according to claim 1, wherein the effective amount of probiotic organism is from approximately 1×10⁵ to approximately 2×10⁵ organisms per confection.

4. A soft, chewable confection according to claim 1, wherein the effective amount of probiotic organism is at least 2×10⁵ organisms per confection.

5. A soft, chewable confection according to claim 1, wherein the probiotic organism is selected from the group consisting of Lactobacillus, Bifidobacteria, Lactococcus, and Streptococcus, and combinations thereof.

6. A soft, chewable confection according to claim 1, wherein the probiotic organism is Lactobacillus acidophilus, Streptococcus thermophilus, or Lactobacillus bifidus, or combinations thereof.

7. A soft, chewable confection containing an active ingredient, wherein the active ingredient consists essentially of an effective amount of at least one probiotic organism and an effective amount of at least one prebiotic, wherein the confection is obtained without using high heat or dehydration.

8. A soft, chewable confection according to claim 7, wherein the confection contains no dairy.

9. A soft, chewable confection according to claim 7, wherein the effective amount of probiotic organism is from approximately 1×10⁵ to approximately 2×10⁵ organisms per confection.

10. A soft, chewable confection according to claim 7, wherein the effective amount of probiotic organism is at least 2×10⁵ organisms per confection.

11. A soft, chewable confection according to claim 7, wherein the probiotic organism is selected from the group consisting of Lactobacillus, Bifidobacterium, Lactococcus, and Streptococcus, and combinations thereof.

12. A soft, chewable confection according to claim 7, wherein the probiotic organism is Lactobacillus acidophilus, Lactobacillus bulgaricus, Lactobacillus rhamnosus, Streptococcus thermophilus, or Bifidobacterium bifidum, or combinations thereof.

13. A soft, chewable confection according to claim 7, wherein the prebiotic is derived from inulin, chicory or honey.

14. A soft, chewable confection according to claim 7, wherein the prebiotic is derived from honey.

15. A soft, chewable confection containing an active ingredient, wherein the active ingredient consists essentially of an effective amount of at least one probiotic organism and a nutrient contained in a glycoprotein matrix, or an enzyme, or both, wherein the confection is obtained without using high heat or dehydration.

16. A soft, chewable confection according to claim 15, wherein the confection contains no dairy.

17. A soft, chewable confection according to claim 15, wherein the effective amount of probiotic organism is from approximately 1×10⁵ to approximately 2×10⁵ organisms per confection.

18. A soft, chewable confection according to claim 15, wherein the effective amount of probiotic organism is at least 2×10⁵ organisms per confection.

19. A soft, chewable confection according to claim 15, wherein the probiotic organism is selected from the group consisting of Lactobacillus, Bifidobacterium, Lactococcus, and Streptococcus, and combinations thereof.

20. A soft, chewable confection according to claim 15, wherein the probiotic organism is Lactobacillus acidophilus, Lactobacillus bulgaricus, Lactobacillus rhamnosus, Streptococcus thermophilus, or Bifidobacterium bifidum, or combinations thereof.

21. A soft, chewable confection according to claim 15, wherein the nutrient contained in a glycoprotein matrix is coenzyme Q₁₀, L-carnitine, or alpha lipoic acid, or a combination thereof.

22. A soft, chewable confection according to claim 15, wherein the enzyme is lactase or papain, or a combination thereof.

23. A soft, chewable confection according to claim 15, wherein the nutrient contained in a glycoprotein matrix is coenzyme Q₁₀, L-carnitine, alpha lipoic acid, or a combination thereof, and the enzyme is lactase or papain, or a combination thereof.

24. A soft, chewable confection containing an active ingredient, wherein the active ingredient consists essentially of at least one probiotic organism, at least one prebiotic, and a nutrient contained in a glycoprotein matrix, or an enzyme, or both, wherein the confection is obtained without using high heat or dehydration.

25. A soft, chewable confection according to claim 24, wherein the confection contains no dairy.

26. A soft, chewable confection according to claim 24, wherein the effective amount of probiotic organism is from approximately 1×10⁵ to approximately 2×10⁵ organisms per confection.

27. A soft, chewable confection according to claim 24, wherein the effective amount of probiotic organism is at least 2×10⁵ organisms per confection.

28. A soft, chewable confection according to claim 24, wherein the probiotic organism is selected from the group consisting of Lactobacillus, Bifidobacterium, Lactococcus, and Streptococcus, and combinations thereof.

29. A soft, chewable confection according to claim 24, wherein the probiotic organism is Lactobacillus acidophilus, Lactobacillus bulgaricus, Lactobacillus rhamnosus, Streptococcus thermophilus, or Bifidobacterium bifidum, or combinations thereof.

30. A soft, chewable confection according to claim 24, wherein the prebiotic is inulin, chicory or honey.

31. A soft, chewable confection according to claim 24, wherein the prebiotic is honey.

32. A soft, chewable confection according to claim 24, wherein the nutrient contained in a glycoprotein matrix is coenzyme Q₁₀, L-carnitine, or alpha lipoic acid, or a combination thereof.

33. A soft, chewable confection according to claim 24, wherein the enzyme is lactase or papain, or a combination thereof.

34. A soft, chewable confection according to claim 24, wherein the nutrient contained in a glycoprotein matrix is coenzyme Q₁₀, L-carnitine, or alpha lipoic acid, or a combination thereof, and the enzyme is lactase or papain, or a combination thereof.

35. A method for enhancing immune function of a host in need thereof comprising administering an effective amount of a confection according to claim 1.

36. A method for enhancing immune function of a host in need thereof comprising administering an effective amount of a confection according to claim 7.

37. A method for enhancing immune function of a host in need thereof comprising administering an effective amount of a confection according to claim 15.

38. A method for enhancing immune function of a host in need thereof comprising administering an effective amount of a confection according to claim 24.

39. A method for providing probiotic organisms to a lactose intolerant host in need thereof comprising administering an effective amount of a soft, chewable confection containing an active ingredient, wherein the active ingredient consists essentially of an effective amount of at least one probiotic organism, wherein the confection is obtained without using high heat or dehydration, and wherein the confection contains no dairy.

40. A method for providing probiotic organisms to a lactose intolerant host in need thereof comprising administering an effective amount of a soft, chewable confection containing an active ingredient, wherein the active ingredient consists essentially of an effective amount of at least one probiotic organism, and an effective amount of lactase, wherein the confection is obtained without using high heat or dehydration.
